(19) United States
(12) Patent Application Publication
(10) Pub. No.: US 2023/0321624 A1
(43) Pub. Date: Oct. 12, 2023

(54) APPARATUS AND METHOD FOR CONVERTING TOXIC GAS OF STERILIZATION PROCESSES TO BENIGN SUBSTANCES

(71) Applicant: BOARD OF TRUSTEES OF THE UNIVERSITY OF ARKANSAS, Little Rock, AR (US)

(72) Inventors: Anindya Ghosh, Little Rock, AR (US); Alexandru S. Biris, Little Rock, AR (US)

(21) Appl. No.: 18/132,523

(22) Filed: Apr. 10, 2023

Related U.S. Application Data
(60) Provisional application No. 63/328,792, filed on Apr. 8, 2022.

(51) Int. Cl. B01J 12/00; B01J 19/24; B01J 35/04; B01J 21/10; B01J 23/75; B01J 20/20; B01J 20/30; B01J 20/28; A61L 2/20; C07D 317/38; B01D 53/86; B01D 53/75; B01D 53/82; B01D 53/72; B01D 53/04

(52) U.S. Cl. CPC B01J 12/007; B01J 19/245; B01J 35/04; B01J 21/10; B01J 23/75; B01J 20/205; B01J 20/3085; B01J 20/28059; B01J 20/28061; A61L 2/206; C07D 317/38; B01D 53/8668; B01D 53/75; B01D 53/82; B01D 53/72; B01D 53/04; A61L 2202/24; A61L 2101/44

(57) ABSTRACT

An apparatus for converting a toxic gas to benign substances comprises a housing characterized with multi-stages including a first stage, a second stage, a third stage and a fourth stage coupled to one another in sequence, wherein the first stage comprises a catalytic system configured to convert the toxic gas into its derivatives; the second stage comprises a carbonaceous fibrous material adapted to capture the remaining toxic gas and the derivatives; the third stage comprises at least one oxidizer to oxidize the remaining toxic gas to benign substances including CO₂ and water; and the fourth stage comprises a scrubber configured to remove all of volatile organic compounds or water molecules generated as part of the first and third stages.

### CROSS-REFERENCE TO RELATED PATENT APPLICATION

This application claims priority to and the benefit of U.S. Provisional Application Ser. No. 63/328,792, filed Apr. 8, 2022, which is incorporated herein by reference in its entirety.

### FIELD OF THE INVENTION

The present invention relates generally to material processing, and more particularly to apparatus and method for converting a toxic gas of sterilization processes to benign substances.

### BACKGROUND OF THE INVENTION

The background description provided herein is for the purpose of generally presenting the context of the present invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the present invention.

Ethylene oxide (EtO) is a gas that is highly efficacious in destroying the membranes of bacteria or other cells, and represents one of the most desired methods for sterilization, in particular for polymeric devices that cannot withstand gamma irradiation or other oxidative processes (NO₂). EtO works through the process of Alkylation of the DNA, RNA or proteins that compose the cells and bacteria. EtO is simple to use and has a high ability to penetrate pores and low dimension morphological voids that make up various medical devices of high porosity. Another major advantage of EtO is the fact that the “load” or volume of medical devices to be sterilized can be very flexible, ranging from one to units such as pallets. EtO's main advantage is its ability to sterilize heat or moisture sensitive medical devices and equipment. Overall, the sterilization process using EtO is simple, reliable and proven. In spite of these positive attributes, EtO is highly toxic and has the risk of being carcinogenic, neurotoxic, and a reproductive toxin. However, for many medical devices and medical equipment there is no other method of sterilization that can accurately and safely sterilize them. Certain polymers, drugs or molecules cannot withstand exposure to gamma irradiation, temperature or other gasses such as NO₂ that are more oxidative and therefore could destroy the chemical structures of the devices. Over the years, a number of improvements have been made to lower the temperature, increase efficiency and optimization of the sterilization cycle. Regardless, these methods continue to result in releasing EtO into the environment. Additionally, different systems are used to reduce the levels of ethylene oxide contained in gaseous mixtures; some of these methods react ethylene oxide with acidic solutions to form ethylene glycol, a non-toxic product that can be disposed of easily. Of these known methods which involve reacting the gas with acidic solutions, the results remain suboptimal since they merely reduce ethylene oxide levels without completely eliminating all residues; furthermore, they involve a lengthy procedure, sometimes with several reducing cycles, leading to a significant waste of energy.

There is an increasing need to accelerate the sterilization cycle while preserving the environment and ensuring the health and safety of workers and consumers. This leads to an increasing urgency for improved systems to remove ethylene oxide (completely) from gaseous mixtures. New approaches must eliminate, as quickly as possible, all traces of potentially harmful ethylene oxide.

### SUMMARY OF THE INVENTION

One of the objectives of this invention is to provide system and method that can eliminate EtO fumes from the sterilization process as quickly as possible, along with all traces of potentially harmful ethylene oxide. The new approach has the potential to entirely remove EtO used during various sterilization cycles. In addition, the new approach is a zero-emission process that does not generate any byproducts besides CO₂ and water. Accordingly, by utilizing the new approach in the sterilization industry, huge amounts of EtO would no longer be released into the environment but rather be destroyed within the novel and unique cartridge system.

Specifically, in one aspect, the invention relates to an apparatus for converting a toxic gas to benign substances. The apparatus comprises a housing characterized with multi-stages including a first stage, a second stage, a third stage and a fourth stage coupled to one another in sequence. The first stage comprises a catalytic system configured to convert the toxic gas into its byproducts. The second stage comprises a carbonaceous fibrous material adapted to capture the remaining toxic gas and the byproducts. The third stage comprises at least one oxidizer to oxidize the remaining toxic gas to benign substances including CO₂ and water. The fourth stage comprises a scrubber configured to remove all of volatile organic compounds or water molecules generated as part of the first and third stages.

In one embodiment, the catalytic system comprises metal catalysts, metal oxide catalysts, supported on metal oxides, zeolites, graphitic materials, Lewis acidic catalysts, bases such as amines, halides, acetates, oxides, nitrites, ammonium, and/or phosphonium.

In one embodiment, the catalytic system comprises metallic or oxides of Co, Fe, Co/Fe, Ni, Co/Mo, Pt, W, supported over MgO, CaCO₃, CaO, zeolites, graphitic nanostructures, SiO₂ other forms of oxides with various compositions and ratios, wherein the ratios range from 0.001 to 99 wt. %.

In one embodiment, the catalytic system is in the form of powders with an average size ranging from nanometers to centimeters, and/or beds of porosities with an average pore size ranging from nanometers to centimeters.

In one embodiment, the catalytic system comprises an active porous catalytic bed formed of a metal oxide nanocluster supported on magnesia (MgO).

In one embodiment, the metal oxide nanocluster comprises cobalt tetraoxide (Co₃O₄) or other forms of cobalt oxides.

In one embodiment, a ratio of Co₃O₄ or other forms of Cobalt oxide to MgO is in a range from 0.01 wt % to 99.99 wt %.

In one embodiment, the first stage further comprises carbon dioxide (CO₂) being introduced into the first stage at a temperature ranging from room temperature to over 100° C.

In one embodiment, the carbonaceous fibrous material is decorated with amine functionalization configured to trap the remaining toxic gas and the derivatives through surface bonding.

In one embodiment, the carbonaceous fibrous material is decorated with amine NH₂ functional chemical groups including primary and secondary amine groups pyridinic, imidazole, and/or with zinc oxide (ZnO) or alumina (Al₂O₃) nanostructures, CaO, CaCO₃, MgO, Ti₂ and derivatives, SiO₂, or zeolites.

In one embodiment, the carbonaceous fibrous material comprises carbon nanofibers having a surface area in a range of 1-2000 m²/g, preferably in a range of 4-200 m²/g.

In one embodiment, the second stage further comprises Lewis acidic catalysts including alumina or zinc salts, metal catalysts, metal oxide catalysts, supported on metal oxides, zeolites, graphitic materials, Lewis acids catalysts, bases such as amines, halides, acetates, oxides, nitrites, ammonium, and/or phosphonium.

In one embodiment, the second stage further comprises polymeric, cellulose or other organic and inorganic porous systems that can be functionalized with chemical group functionalities, wherein the other organic and inorganic porous systems include chitosan, starch, Xanthan, alginate, polyvinyl alcohol, alumina, ZnO, SiO2, MgO, CaO, CaCo3, and/or zeolites.

In one embodiment, the at least one oxidizer comprises a solid oxidizer including persulphate or perborate.

In one embodiment, the third stage further comprises metal salts adapted to induce Fenton type chemistry along with the solid oxidizer.

In one embodiment, the metal salts includes cobalt oxide or iron oxide or iron sulfate particles

In one embodiment, the at least one oxidizer and the metal salts are made into a porous particulate bed supported over an alumina oxide structure.

In one embodiment, the scrubber comprises high surface area activated charcoal or graphitic structures, carbon nanostructures of various shapes and sizes (graphene, fibers, nanotubes, plates), porous structures decorated with such materials, graphite, cellulose, Chitosan, starch, Xanthan, alginate, polyvinyl alcohol, and/or polyurethanes.

In one embodiment, the fourth stage further comprises adsorbing media that capture the remaining toxic gas and the derivatives and can be functionalized with various functional chemical groups.

In one embodiment, the adsorbing media comprise zeolites, graphitic materials, polymeric structures, metal/metal oxides, cellulose, Lewis acidic catalysts, organic or inorganic porous systems, or a combination of them.

In one embodiment, the toxic gas comprises ethylene oxide, propylene oxide, ozone, nitric oxides, NOx, volatile organic carbons, CO, or sox.

In another aspect of the invention, the method for converting a toxic gas into benign substances comprises catalytically converting the toxic gas into its byproducts in a first stage; capturing the remaining toxic gas and the byproducts in a second stage; oxidizing the remaining toxic gas to benign substances including CO₂ and water in a third stage; and removing, from a fourth stage, all of volatile organic compounds or water molecules generated as part of the first and third stages.

In one embodiment, the first stage comprises a catalytic system comprising metal catalysts, metal oxide catalysts, supported on metal oxides, zeolites, graphitic materials, Lewis acidic catalysts, bases such as amines, halides, acetates, oxides, nitrites, ammonium, and/or phosphonium.

In one embodiment, the catalytic system comprises metallic or oxides of Co, Fe, Co/Fe, Ni, Co/Mo, Pt, W, supported over MgO, CaCO₃, CaO, graphitic nanostructures, SiO₂ with various compositions and ratios, wherein the ratios range from 0.001 to 99 wt. %.

In one embodiment, the catalytic system is in the form of powders with an average size ranging from nanometers to centimeters, and/or beds of porosities with an average pore size ranging from nanometers to centimeters.

In one embodiment, the catalytic system comprises an active porous catalytic bed formed of a metal oxide nanocluster supported on magnesia (MgO).

In one embodiment, the metal oxide nanocluster comprises cobalt tetraoxide (Co₃O₄).

In one embodiment, a ratio of Co₃O₄ to MgO is in a range from 0.01 wt % to 99.99 wt %.

In one embodiment, the method further comprises intruding carbon dioxide (CO₂) into the first stage at a temperature ranging from room temperature to over 100° C.

In one embodiment, the second stage comprises a carbonaceous fibrous material decorated with amine functionalization configured to trap the remaining toxic gas and the derivatives through surface bonding.

In one embodiment, the carbonaceous fibrous material is decorated with amine NH₂ functional chemical groups including primary and secondary amine groups pyridinic, imidazole, and/or with zinc oxide (ZnO) or alumina (Al₂O₃) nanostructures, CaO, CaCO₃, MgO, Ti₂ and derivatives, SiO₂, or zeolites.

In one embodiment, the carbonaceous fibrous material comprises carbon nanofibers having a surface area in a range of 1-200 m²/g, preferably in a range of 40-100 m²/g.

In one embodiment, the second stage further comprises Lewis acidic catalysts including alumina or zinc salts, metal catalysts, metal oxide catalysts, supported on metal oxides, zeolites, graphitic materials, Lewis acids catalysts, bases such as amines, halides, acetates, oxides, nitrites, ammonium, and/or phosphonium.

In one embodiment, the second stage further comprises polymeric, cellulose or other organic and inorganic porous systems that can be functionalized with chemical group functionalities, wherein the other organic and inorganic porous systems include chitosan, starch, Xanthan, alginate, polyvinyl alcohol, alumina, ZnO, SiO₂, MgO, CaO, CaCO₃, and/or zeolites.

In one embodiment, the third stage comprises at least one oxidizer comprising a solid oxidizer including persulphate or perborate.

In one embodiment, the third stage further comprises metal salts adapted to induce Fenton type chemistry along with the solid oxidizer.

In one embodiment, the metal salts includes cobalt oxide or iron oxide or iron sulfate particles

In one embodiment, the at least one oxidizer and the metal salts are made into a porous particulate bed supported over an alumina oxide structure.

In one embodiment, the fourth stage comprises a scrubber comprising high surface area activated charcoal or graphitic structures capable of being recycled.

In one embodiment, the fourth stage further comprises adsorbing media that capture the remaining toxic gas and the derivatives and can be functionalized with various functional chemical groups.

In one embodiment, the adsorbing media comprise zeolites, graphitic materials, polymeric structures, metal/metal oxides, cellulose, Lewis acidic catalysts, organic or inorganic porous systems, or a combination of them.

In one embodiment, the toxic gas comprises ethylene oxide or propylene oxide.

These and other aspects of the invention will become apparent from the following description of the preferred embodiment taken in conjunction with the following drawings, although variations and modifications therein may be affected without departing from the spirit and scope of the novel concepts of the invention.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment.

FIG. 1 shows schematically an apparatus for converting a toxic gas to benign substances according to certain embodiments of the invention.

FIG. 2 shows FT-IR spectrum of propylene carbonate. The peak at 1797 cm⁻¹ is characteristic of propylene carbonate.

FIG. 3 shows gas chromatogram of propylene carbonate. The peak at 6.75 min is due to propylene carbonate.

FIG. 4 shows GC mass spectrum of propylene carbonate.

FIG. 5 shows the product propylene carbonate isolated and characterized using FT/IR and GC/MS.

FIG. 6 shows certain exemplary nanosized catalysts.

FIG. 7 shows schematically catalytic reaction of propylene oxide.

FIG. 8 shows schematically catalytic reaction of ethylene oxide.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting and/or capital letters has no influence on the scope and meaning of a term; the scope and meaning of a term are the same, in the same context, whether or not it is highlighted and/or in capital letters. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only and in no way limits the scope and meaning of the invention or of any exemplified term. Likewise, the invention is not limited to various embodiments given in this specification.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below can be termed a second element, component, region, layer or section without departing from the teachings of the invention.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” to another feature may have portions that overlap or underlie the adjacent feature.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top”, may be used herein to describe one element's relationship to another element as illustrated in the figures. It will be understood that relative terms are intended to encompass different orientations of the device in addition to the orientation shown in the figures. For example, if the device in one of the figures is turned over, elements described as being on the “lower” side of other elements would then be oriented on the “upper” sides of the other elements. The exemplary term “lower” can, therefore, encompass both an orientation of lower and upper, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present invention, and will not be interpreted in an idealized or overly formal sense unless expressly so defined herein.

As used herein, the terms “comprise” or “comprising”, “include” or “including”, “carry” or “carrying”, “has/have” or “having”, “contain” or “containing”, “involve” or “involving” and the like are to be understood to be open-ended, i.e., to mean including but not limited to.

As used herein, the phrase “at least one of A, B, and C” should be construed to mean a logical (A or B or C), using a non-exclusive logical OR. It should be understood that one or more steps within a method may be executed in different order (or concurrently) without altering the principles of the invention.

As used herein, terms such as “about”, “approximately”, “generally”, “substantially”, and the like unless otherwise indicated mean within 20 percent, preferably within 10 percent, preferably within 5 percent, and even more preferably within 3 percent of a given value or range. Numerical quantities given herein are approximate, meaning that the term “about”, “approximately”, “generally”, or “substantially” can be inferred if not expressly stated.

The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

Ethylene oxide (EtO) is widely used in hospitals as a gaseous sterilant for heat-sensitive medical items, surgical instruments and other objects and fluids that come in contact with biological tissues. Large sterilizers are found in central supply areas of most hospitals and commercial medical device producers; smaller sterilizers are found in clinics, operating rooms, tissue banks and research facilities. Since 2016, EtO has been identified by EPA as a carcinogen. The National Institute for Occupational Safety and Health (NIOSH) and The US Occupational Safety and Health Administration (OSHA) issue guidance that limit occupational exposures to EtO.

There are three reasons that it is difficult for the medical industry to replace EtO in the sterilization process: 1) a viable replacement has not been identified for biomedical devices comprised of polyurethane derivatives; 2) EtO sterilization accounts for 50% of the medical sterilization market of which 20% of the devices have no alternative method; and, 3) portability and cost of EtO sterilization methods allows accessibility to a wide range of applications, environments, and regions.

EtO sterilization is a priority issue for the FDA which issued a challenge in 2019 seeking to identify safe and effective methods or technologies for medical devices that do not rely on ethylene oxide or to create innovations which reduce or eliminate emissions from the EtO sterilization process. The first challenge had four participants with a total of 5 alternative methods to be researched. Unfortunately, none of these methods have shown favorable results for use on polyurethane, the base for many new innovative medical devices that NuShores and others have in development. EtO remains the most effective process for sterilization of medical devices that include polymers.

Previous attempts to solve the EtO emission problem have focused on methods involving absorption, combustion, biodegradation, or simply identifying the minimal amount of EtO needed while still achieving safety and sterilization protocols. These efforts have largely been proven to be hazardous, ineffective, or costly.

In view of the foregoing, we have developed a multi stage reactor/cartridge system that can be attached to any sterilization chambers that used EtO for sterilization and which degrades the EtO gas into non-toxic byproducts. The process uses complex chemical staged reactions that degrades and captures the byproducts generated from the EtO used during the sterilization process.

Specifically, the invention in certain aspects relates to an apparatus and a method/process for converting a toxic gas to benign substances. In one embodiment, the toxic gas comprises ethylene oxide, propylene oxide, ozone, nitric oxides, NOx, volatile organic carbons, CO, or sox. The apparatus can be in the form of cartridge operably attached behind an EtO sterilizer.

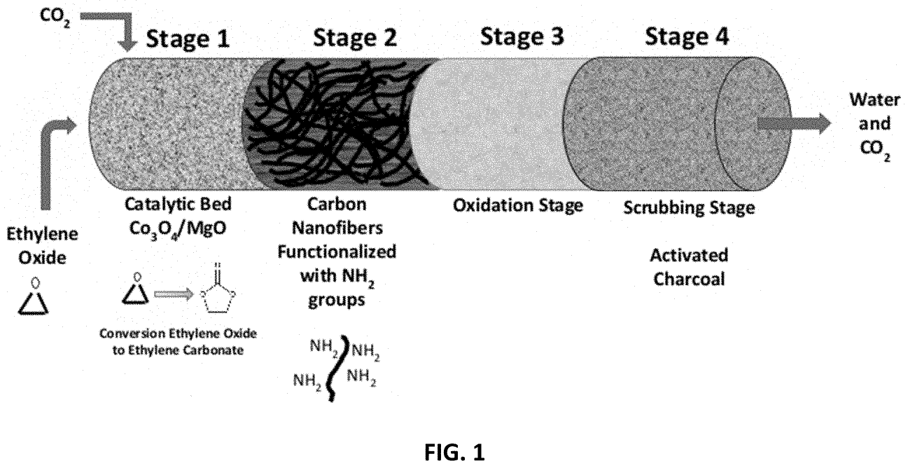

As shown in FIG. 1, the apparatus comprises a housing characterized with multi-stages including a first stage, a second stage, a third stage, and a fourth stage coupled to one another in sequence. The first stage comprises a catalytic system configured to convert the toxic gas into its byproducts. The second stage comprises a carbonaceous fibrous material adapted to capture the remaining toxic gas and the byproducts. The third stage comprises at least one oxidizer to oxidize the remaining toxic gas to benign substances including CO₂ and water. The fourth stage comprises a scrubber configured to remove all of volatile organic compounds or water molecules generated as part of the first and third stages.

The method for converting a toxic gas into benign substances comprises catalytically converting the toxic gas into its byproducts in a first stage; capturing the remaining toxic gas and the byproducts in a second stage; oxidizing the remaining toxic gas to benign substances including CO₂ and water in a third stage; and removing, from a fourth stage, all of volatile organic compounds or water molecules generated as part of the first and third stages.

In one embodiment, the catalytic system comprises metal catalysts, metal oxide catalysts, supported on metal oxides, zeolites, graphitic materials, Lewis acidic catalysts, bases such as amines, halides, acetates, oxides, nitrites, ammonium, and/or phosphonium. The metal catalysts, etc. So the actual catalytic structure is composed of catalytically active metal and metal oxide (generally nanostructures) supported on large surface area metal oxides supports.

In one embodiment, the catalytic system comprises metallic or oxides of Co, Fe, Co/Fe, Ni, Co/Mo, Pt, W, supported over MgO, CaCo₃, CaO, graphitic nanostructures, SiO₂ with various compositions and ratios.

In one embodiment, the catalytic system is in the form of powders with an average size ranging from nanometers to centimeters, e.g., from 1 nm to 10 cm, and/or beds of porosities with an average pore size ranging from nanometers to centimeters, e.g., from 1 nm to 10 cm.

In one embodiment, the catalytic system comprises an active porous catalytic bed formed of a metal oxide nanocluster supported on magnesia (MgO).

In one embodiment, the metal oxide nanocluster comprises cobalt tetraoxide (Co₃O₄).

In one embodiment, a ratio of Co₃O₄ to MgO is in a range from 0.01 wt % to 99.99 wt %. In certain embodiments, the ratio is in a range of 0.01-20 wt %, 20-40 wt %, 40-60 wt %, 60-80 wt %, or 80-99.99 wt %.

In one embodiment, the first stage further comprises carbon dioxide (CO₂) being introduced into the first stage at a temperature ranging from room temperature to over 100° C.

In one embodiment, the carbonaceous fibrous material is decorated with amine functionalization configured to trap the remaining toxic gas and the derivatives through surface bonding.

In one embodiment, the carbonaceous fibrous material is decorated with amine NH₂ functional chemical groups including primary and secondary amine groups pyridinic, imidazole, and/or with zinc oxide (ZnO) or alumina (Al₂O₃) nanostructures, CaO, CaCO₃, MgO, Ti₂ and derivatives, SiO₂, or zeolites.

In one embodiment, the carbonaceous fibrous material comprises carbon nanofibers having a surface area in a range of 1-200 m²/g. In certain embodiments, the surface area is in a range of 1-20 m²/g, 20-40 m²/g, 40-60 m²/g, 60-80 m²/g, 80-100 m²/g, 100-120 m²/g, 120-140 m²/g, 140-160 m²/g, 160-180 m²/g, or 180-200 m²/g. Preferably, the surface area is in a range of 40-100 m²/g.

In one embodiment, the second stage further comprises Lewis acidic catalysts including alumina or zinc salts, metal catalysts, metal oxide catalysts, supported on metal oxides, zeolites, graphitic materials, Lewis acids catalysts, bases such as amines, halides, acetates, oxides, nitrites, ammonium, and/or phosphonium.

In one embodiment, the second stage further comprises polymeric, cellulose or other organic and inorganic porous systems that can be functionalized with chemical group functionalities, wherein the other organic and inorganic porous systems include chitosan, starch, Xanthan, alginate, polyvinyl alcohol, alumina, ZnO, SiO2, MgO, CaO, CaCo3, and/or zeolites.

In one embodiment, the at least one oxidizer comprises a solid oxidizer including persulphate or perborate.

In one embodiment, the third stage further comprises metal salts adapted to induce Fenton type chemistry along with the solid oxidizer.

In one embodiment, the metal salts includes cobalt oxide or iron oxide or iron sulfate particles

In one embodiment, the at least one oxidizer and the metal salts are made into a porous particulate bed supported over an alumina oxide structure.

In one embodiment, the scrubber comprises high surface area activated charcoal or graphitic structures, carbon nanostructures of various shapes and sizes (graphene, fibers, nanotubes, plates), porous structures decorated with such materials, graphite, cellulose, Chitosan, starch, Xanthan, alginate, polyvinyl alcohol, and/or polyurethanes.

In one embodiment, the fourth stage further comprises adsorbing media that capture the remaining toxic gas and the derivatives and can be functionalized with various functional chemical groups.

In one embodiment, the adsorbing media comprise zeolites, graphitic materials, polymeric structures, metal/metal oxides, cellulose, Lewis acidic catalysts, organic or inorganic porous systems, or a combination of them.

These and other aspects of the present invention are further described in the following section. Without intending to limit the scope of the invention, further exemplary implementations of the present invention according to the embodiments of the present invention are given below. Note that titles or subtitles may be used in the examples for the convenience of a reader, which in no way should limit the scope of the invention. Moreover, certain theories are proposed and disclosed herein; however, in no way should they, whether they are right or wrong, limit the scope of the invention so long as the invention is practiced according to the invention without regard for any particular theory or scheme of action.

### Example 1

### Martial Designs of Catalytically-Active Cartridge System

The high diffusivity of EtO gas through solids makes it an ideal sterilization method, particularly for the growing number of implantable devices that are sensitive or incompatible with sterilization methods that utilize heat, moisture, radiation, or other gasses. Materials degradation is a major sterilization concern, and the EtO method overcomes this issue—especially with polyurethane-based materials. As a sterilization method, EOG is widely documented for its strong microbicidal, virucidal, and fungicidal abilities. However, EtO is a carcinogen identified by EPA and is hazard to workers, patients, staff, and the environment.

We have developed a composite material to compete in the bone filler industry with the potential to address multiple indications. Early non-GLP (good laboratory practices) animal studies suggest our patented technology may offer improved solutions for bone regeneration while lowering healthcare costs, reducing treatment risks and healing times. NuShores' bone filler scaffold technologies offer a number of benefits to date not achieved by currently marketed bone regeneration products, therefore promising to bring better treatment outcomes to millions of people with severe bone injuries. After manufacturing the composite material, EtO is the only method acceptable of sterilization.

A process is needed to neutralize the EtO gasses so that the FIAB (factory in a box) may be used in remote or austere locations. We therefore design a multi-stage (shown here 4 stages) catalytic cartridge-based filtration/scrubber device that converts the toxic gasses to benign components. The filtration/scrubber device can be housed in a cartridge form and potentially added to other EtO sterilization equipment. The novel approach is a cost-effective solution that is safe for humans and the environment alike, while utilizing the known benefits of EtO sterilization, and offers the possibility of removing all toxic aspects of use of EtO, enabling the emerging biomedical polymer-based device industry. Sterilization could be available at any dental or medical unit without concerns for the environment or for the safety of personnel. Eliminating concerns with EtO usage and given the limitations of other sterilization techniques, EtO could gain market share and could become the method of choice for reliable sterilization needs.

This invention discloses a multi stage reactor that can completely or partially remove toxic EtO used in sterilization processes by catalytically convert it into less or non-toxic compounds or scrubbing it or its byproducts such that the removal goal is completed. For this we disclose a multi stage process, that include a stage for catalytically converting it to other byproducts, a stage for oxidizing it, a stage of filtration and attachment, a stage for scrubbing it or its byproducts. We also disclose the combination of such a device with water steam to enhance the collection of EtO.

FIG. 1 shows schematically a cartage system including first, second, third and fourth stages according to embodiments of the invention. Each stages may be fitted with appropriate pressure gauge and temperature controller to read the pressure and temperatures. Additionally, pressure and temperature sensors may be used for monitoring the pressure and temperature. The first stage includes a catalytically active system that converts ethylene oxide to ethylene carbonate. The second stage includes a high surface area carbonaceous fibrous material with high density amine functionalization for trapping the ethylene oxide as well as ethylene carbonate through surface bonding. The third stage includes a metal catalyst and a solid oxidizer to oxidize any remaining ethylene oxide to benign products (such as CO₂ and water). The fourth stage has a high efficiency scrubber that removes all the volatile organic compounds. It should be noted that the term “stage”, used in the disclosure, can be a chamber or reactor. Certain exemplary embodiments of the four stages of the invention are described below.

Material Stage 1: The first stage has a highly active porous catalytic bed formed of a metal oxide nanocluster supported on magnesia (MgO). In one exemplary embodiment, cobalt tetraoxide (Co₃O₄) is adapted as the active catalytic nanosystem and then the ratio of the Co₃O₄ to MgO is varied to study and enhance the catalytic conversion. The ratio of Co₃O₄ to MgO can be changed from 0 to 99.99 wt %. The catalyst amount is determined based on the amount of EtO used for sterilization. As part of the effort to increase conversion reaction efficiency, certain amounts of CO₂ are introduced into the reaction stage at various temperatures, for example, from room temperature to over 100° C.

Any catalytic system can be used to convert EtO to other molecules. In certain embodiments, the catalytic system includes, but not limited to, metal (transition or non-transition metals) catalysts, metal oxide catalysts, supported on metal oxides, zeolites, graphitic materials, any Lewis acidic catalysts, bases such as amines, halides, acetates, oxides, nitrites, ammonium, phosphonium, and so on. The catalytic system can be in the form of powders with an average size in a range from an order of nanometers to an order of centimeters, and/or beds of various shapes and sizes of porosities with pore sizes that vary from nanometers to centimeters.

Material Stage 2: The second stage includes highly dense carbon nanofibers with a high surface area of 40-100 m²/g that are decorated with amine NH₂ functional chemical groups. Amine groups react covalently with the EtO groups that might come out of the first stage (Stage 1). This approach is useful for capturing any EtO molecules escaping from the catalytic reaction Stage 1. The EtO is efficiently captured through strong bonding to the high surface area of amine decorated carbon nanofibers and once attached, these molecules do not escape or detach. To further increase efficiency of EtO molecule capture, the temperature at the second stage can be of 25, 40, or 90° C. To further enhance the reactivity of the amine groups, the addition of Lewis acids such as alumina or zinc salts are included. Finally, carbon nanofibers are decorated with ZnO or alumina oxide nanostructures.

In one embodiment, the second stage includes the materials that can capture EtO and its derivatives. The second stage may also include, but not limited to, metal (transition or non-transition metals) catalysts, metal oxide catalysts, supported on metal oxides, zeolites, graphitic materials, any Lewis acids catalysts, bases such as amines, halides, acetates, oxides, nitrites, ammonium, phosphonium, and the likes. These catalytic systems can be in the form of powders (nanometers to centimeters), beds (of various shapes and sizes) of porosities that vary from nanometers to centimeters. The second stage may further include polymeric (natural or synthetic), cellulose or other organic and inorganic porous systems that can be functionalized with chemical group functionalities. The second stage may also include multi component organic-inorganic phases of various dimensions and ratios.

In certain embodiments, the graphitic nanomaterials are synthesized over various catalytic systems. A variety of nanostructural materials are synthesized to include single wall carbon nanotubes, multiwall carbon nanotubes nanofibers as well as graphene. Further, various catalytic systems include metallic or oxides of Co, Fe, Co/Fe, Ni, Co/Mo, Pt, W, supported over MgO, CaCo₃, CaO, graphitic nanostructures, SiO₂ with various compositions and ratios.

In certain embodiments, the carbon nanostructures have an ultimate purity, exceeding 98-99 wt. %. This high purity along with the unique carbon structure is ideal for the use of such nanostructures in complex biochemical reactions. The major advantage that such graphitic nanostructures offer is their ability and relative ease of having various chemical functionalities reliably attached onto their surfaces. Surface chemical functionalization with chemical groups, such as carboxylic, amide, amine, allows these nanomaterials to be used as the active component in various systems used for filtration of complex agents out of water or gas, advanced composites or biomedical applications. In certain embodiments, functionalization of carbon nanotubes, carbon nanospheres, carbon nanofibers with both amine and carboxylic groups is shown. Furthermore, the attachment through chemical functionalization of antibodies, various proteins, plasmids/DNA onto the surface of the graphitic nanomaterials is shown.

In addition, the graphitic nanomaterials are not only decorated with metallic clusters or nano materials composed of transition metals such as cobalt and/or iron, but also with noble metals such as gold and/or silver. The resulting metal-graphitic nanocomposite with different chemistries and morphologies have been used successfully for the filtration of organic impurities from both liquid and gaseous environments. Moreover, the catalytic system may also include various nanostructural composites based on graphitic nanomaterials and Au, Au/Ag, Ti, Pt, Co, NiO—Mn₂O₃/carbon nanocomposites.

Materials Stage 3: Further oxidation takes place in the third stage. In spite of the high efficiency reactivity of the first and second stages, some molecules of EtO or cyclic carbonates that could be generated in the first stage could still escape. In certain embodiments, the oxidizing stage includes metal salts that induce Fenton type chemistry, such as cobalt oxide or iron oxide or iron sulfate particles, along with a solid oxidizer, such as persulphate or perborate. These chemical agents are made into a porous particulate bed supported over an alumina oxide structure. The third stage further fragments the EtO or cyclic carbonate molecules to water and CO₂ byproducts, harmless compared to the original compounds.

The third stage can be formed of any material that can oxidize the EtO molecules or its derivatives.

Materials Stage 4: The fourth stage is configured to scrub any organic or water molecules generated as part of the first and third stages (Stages 1 and 3). Specifically, high surface area activated charcoal or graphitic structures that are capable of being recycled. This chemistry and such materials have already been developed and tested for such purposes.

The fourth stage can be composed of any adsorbing media that can capture the EtO or its byproducts and can be functionalized with various functional chemical groups, can have porosities from nanometers to centimeters, can be made out of zeolites, graphitic materials, polymeric structures, metal/metal oxides, cellulose, combination of them, any Lewis acidic catalysts (metals and non-metals), organic and inorganic porous systems, etc.

Material for Housing: In some embodiments, various materials can be used to generate a robust and safe housing environment for each of the four stages. In one embodiment, the housing is formed of stainless steel, which is rather unreactive to the chemicals used in this study and is capable of withstanding both elevated temperatures and pressure.

### Example 2

### The Catalytic Reaction of Epoxide and Carbon Dioxide to Remediate Ethylene and Propylene Oxides

Ethylene oxide is widely used to sterilize different equipment and surfaces. Particularly, it is used to sterilize medical equipment, including heat-sensitive materials such as gloves, plastic syringes, etc. However, ethylene oxide is extremely toxic, mutagenic, and a known carcinogen. Therefore, extreme caution must be taken during the ethylene oxide sterilization process. A lack of suitable technology does not allow us to use ethylene oxide easily during the sterilization process. In order to decontaminate ethylene oxide, we proposed catalytically reacting carbon dioxide in the presence of a catalyst. The desired product is ethylene carbonate which is much less toxic than ethylene oxide. To check the efficacy of the catalysts, we tested the reaction using propylene oxide, as propylene oxide is much safer to handle than ethylene oxide. We are currently working with ethylene oxide to react with carbon dioxide catalytically. We anticipate similar result to produce ethylene carbonate during the reaction as both ethylene oxide and propylene oxide behave similarly in terms of reactivity.

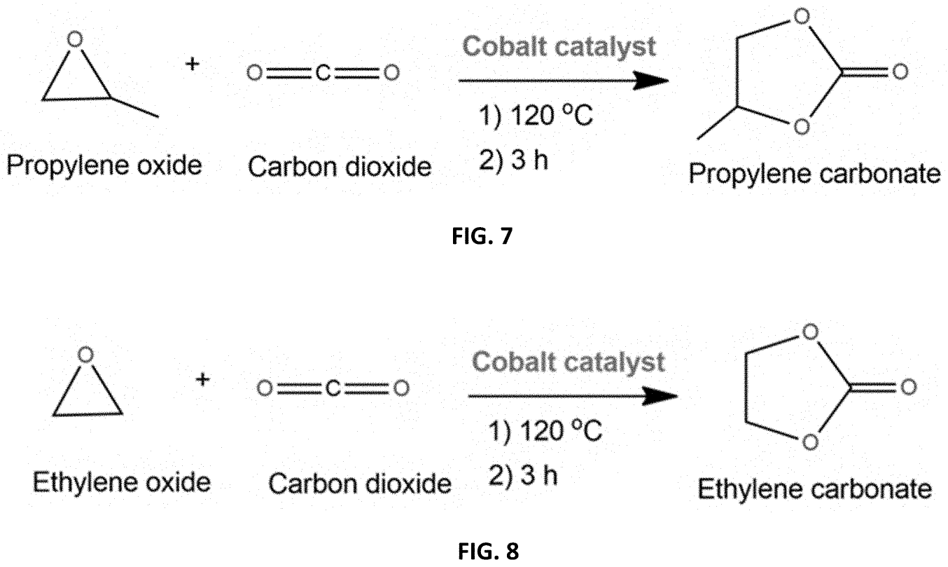

Initial Reaction with Propylene Oxide: In the exemplary example, propylene oxide was used for this reaction, as shown in FIG. 7. The reaction was performed using a 50-milliliter stainless steel Parr high-pressure reactor. Propylene Oxide (2.08 g, 0.036 mol), cobalt catalyst (15 mg), and co-catalyst (8.6 mg, 0.07 mmol) were mixed into the high-pressure reactor. The reactor was pressurized with CO₂ (Purity 99.9%, Airgas), and the temperature of the reactor was brought to at 120° C. and maintained at that temperature. The final pressure of the reaction vessel read as 300 psi (2.06 MPa). The reaction mixture was stirred in these conditions for 3 h. After the reaction, the reactor was quickly brought to a low temperature using an ice water bath, and pressure was released slowly inside a fume hood. The reaction mixture was collected using dichloromethane for a complete transfer. The catalyst was separated by passing through a small plug of silica gel (2 g, 60-100 mesh) and eluting with dichloromethane. Solvent and excess epoxide were removed at reduced pressure. The isolated yield was calculated by taking the weight of the isolated product.

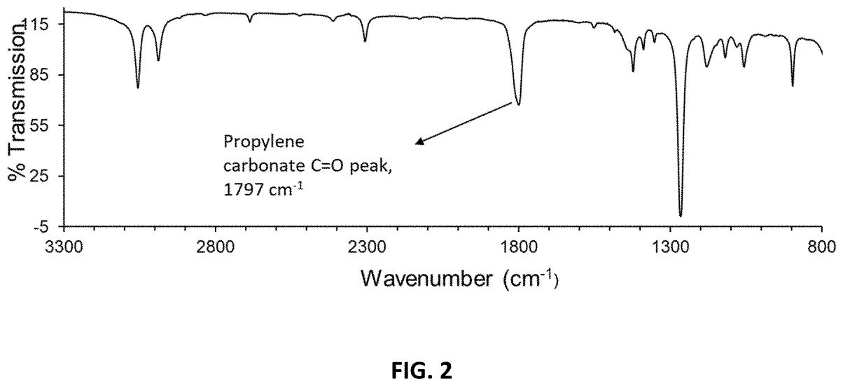

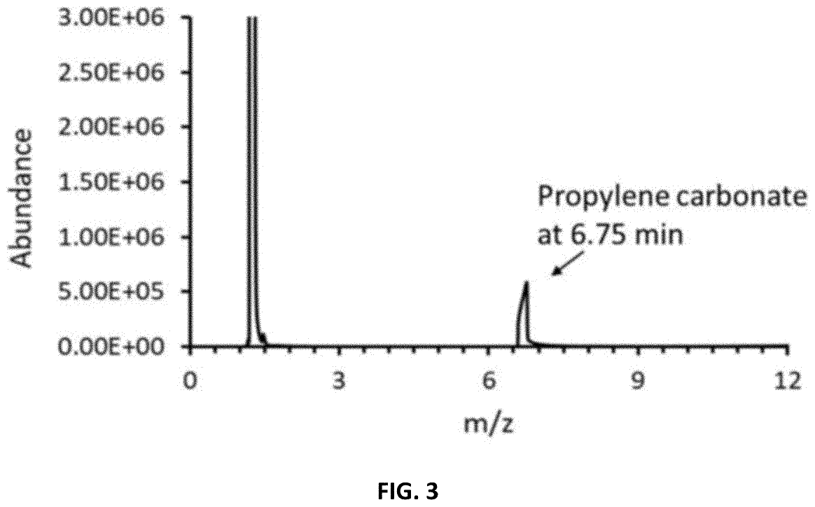

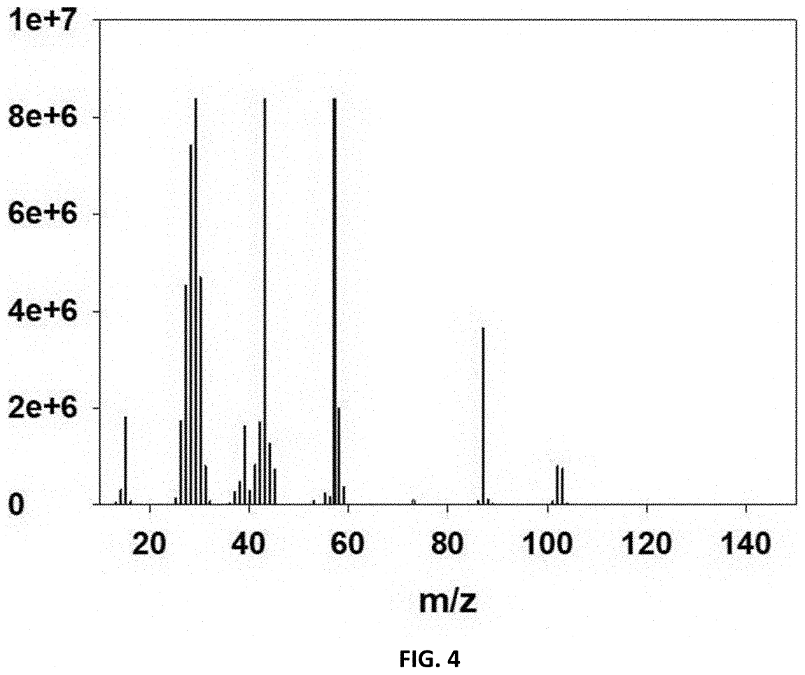

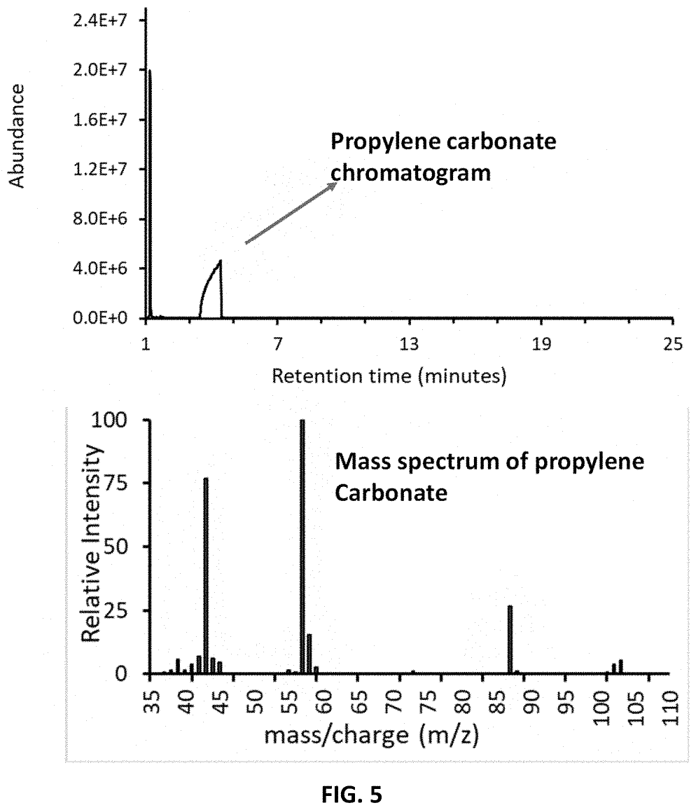

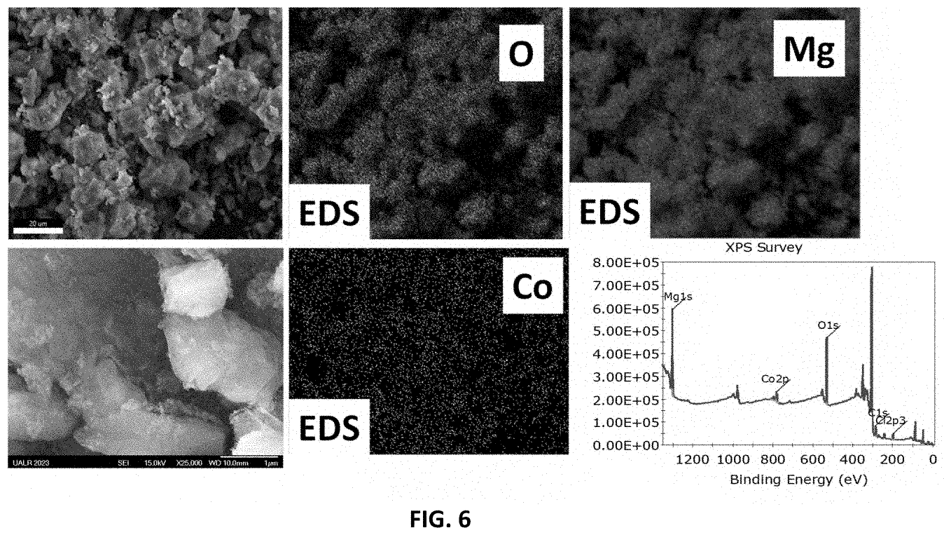

Data Analysis: The isolated propylene carbonate was characterized using Fourier transform infrared spectroscopy (FT/IR) and gas chromatography-mass spectrometry (GC/MS). FIG. 2 shows the FT/IR spectrum of propylene carbonate. The peak at 1797 cm′ is due to the carbonate carbonyl stretching frequency, which is characteristic of propylene carbonate. The GC/MS of the isolated product is checked. The gas chromatogram and the mass spectrum of the propylene carbonate are given in FIGS. 3-5, respectively, which further indicate the presence of the desired propylene carbonate. The peak at 6.75 min is due to propylene carbonate.

Co₃O₄/MgO nanosized catalyst: We synthesized a nanostructural Co₃O₄/MgO catalyst with a ratio of about 5/95 wt. % Co₃O₄/MgO. The catalyst was thermally treated at about 400° C. for about 4 hours. Currently, the catalyst is being characterized by an array of analytical techniques, such SEM/EDS, XRD, and XPS.

This catalyst is evaluated towards EtO catalytical degradation. In one embodiment, cobalt catalyst (15 mg) and co-catalyst (8.6 mg, 0.07 mmol) that were mixed into the high-pressure reactor with PO.

Control Ethylene Oxide: In the first step of the decontamination process, ethylene oxide reacts with carbon dioxide under pressure to convert ethylene oxide to ethylene carbonate.

Ethylene carbonate is much less toxic than ethylene oxide. (FIG. 8).

In certain embodiments, the reaction with different catalyst loading and catalysts are performed, under different reaction conditions, such as temperature, pressure, and time, to optimize the reaction further. Finally, the reaction is performed with ethylene oxide to find the most suitable catalyst and reaction conditions.

The current equipment in use combines sterilization and aeration in the same chamber, or else in a continuous chamber, so that non-stop processing may occur, and potential occupational exposure can be minimized. This process is considered technically complex by industry standards, requiring many layers of security and regulatory practices to compensate for potential toxicity; personnel must be knowledgeable and trained as skilled technicians to properly operate and monitor this process.

The invention discloses a multi stage reactor/cartridge system that can be attached to any sterilization chambers that used EtO for sterilization and which degrades the EtO gas into non-toxic byproducts. The process uses complex chemical staged reactions that degrades and captures the byproducts generated from the EtO used during the sterilization process.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

Some references, which may include patents, patent applications, and various publications, are cited and discussed in the description of this invention. The citation and/or discussion of such references is provided merely to clarify the description of the invention and is not an admission that any such reference is “prior art” to the invention described herein. All references cited and discussed in this specification are incorporated herein by reference in their entireties and to the same extent as if each reference was individually incorporated by reference.

### REFERENCE LIST

[1]. Shintani H. (2017). Ethylene Oxide Gas Sterilization of Medical Devices. Biocontrol science, 22(1), 1-16. https://doi.org/10.4265/bio.22.1
[2]. Anderson, D. A. and Biris, A. S. (2018) Assessment of a novel 3D tunable scaffold technology for bone tissue regeneration of critical sized defects using a goat tibia segmental defect model. Military Health System Research Symposium, presentation at MHSRS 2018
[3]. Sun, B., Wen, M., Ma, X., Zhou, Y., Zhu, Q., Lu, H. (2019). Rapid absorption of ethylene oxide exhaust by catalytic hydration on acid zeolite catalysts. Environmental Technology & Innovation, 15, 100376. https://doi.org/10.1016/j.eti.2019.100376
[4]. Weiss, S., Cogdill, P., Hansen, J. M. (2020). Use of overkill half-cycle qualification data to support reduction of exposure time in validated ethylene oxide sterilization cycles. Biomedical Instrumentation & Technology, 54(s1), 40-44. https://doi.org/10.2345/0899-8205-54.s3.40
[5]. Centers for Disease Control and Prevention. (2016, September 18). Ethylene Oxide Sterilization. Retrieved May 7, 2020 from https://www.cdc.gov/infectioncontrol/guidelines/disinfection/sterilization/ethyleneoxide.html
[6]. Glaser Z R (1979). Ethylene oxide: Toxicology review and field study results of hospital use. Journal of Environmental Pathology and Toxicology, 12: 173-208.
[7]. U.S. Environmental Protection Agency. Evaluation of the Inhalation Carcinogenicity of Ethylene Oxide (CASRN 75-21-8) In Support of Summary Information on the Integrated Risk Information System (IRIS). National Center for Environmental Assessment, Office of Research and Development. Washington, DC. EPA/635/R 16/350Fa. 2016. https://cfpub.epa.gov/ncea/iris/iris_documents/documents/toxreviews/1025tr.pdf
[8]. Jellinek, H. H., & Wang, T. J. (1973). Reaction of nitrogen dioxide with linear polyurethane. Journal of Polymer Science: Polymer Chemistry Edition, 11(12), 3227-3242. https://doi.org/10.1002/pol.1973.170111216
[9]. Parenky, A. C., Wadhwa, S., Chen, H. H., Bhalla, A. S., Graham, K. S., & Shameem, M. (2021). Container Closure and Delivery Considerations for Intravitreal Drug Administration. AAPS PharmSciTech, 22(3), 100. (Table 4). https://doi.org/10.1208/s12249-021-01949-4
[10]. Allied Market Research/Bhardwaj, V., Alhat, A. (February 2022). Sterilization Technologies Market by Type (Chemical and Gas Sterilization, Filtration Sterilization, Ionizing Radiation Sterilization, and Thermal Sterilization) and End User (Pharmaceuticals, Medical Devices, Food and Beverages, Agriculture, Cosmetic, Manufacturing, and Others): Global Opportunity Analysis and Industry Forecast, 2021-2030. https://www.alliedmarketresearch.com/sterilization-technologies-market
[11]. M. (2021, June 3). Medical device sterilization: 4 alternatives to using ethylene oxide. mddionline.com. Retrieved Apr. 5, 2022, from https://www.mddionline.com/sterilization/medical-device-sterilization-4-alternatives-using-ethylene-oxide
[12]. Centers for Disease Control and Prevention. (2016). Ethylene Oxide “Gas” Sterilization Guideline for Disinfection and Sterilization in Healthcare Facilities. Centers for Disease Control and Prevention. https://www.cdc.gov/infectioncontrol/guidelines/disinfection/sterilization/ethylene-oxide.html
[13]. U.S. Department of Health and Human Services. (2020). Pryzbyla, J., Roney, N., Abadin, H., Alman, B., Wohlers, D. W., Barber, L., Diamond, G. L., & Citra, M., Toxicological profile for ethylene oxide: Draft for public comment 1-246.
[14]. Pruneanu, S., Biris, A. R., Pogacean, F., Coroş, M., Kannarpady, G. K., Watanabe, F., & Biris, A. S. (2014). The study of adenine and guanine electrochemical oxidation using electrodes modified with graphene-platinum nanoparticles composites. Electrochimica Acta, 139, 386-393. https://doi.org/10.1016/j.electacta.2014.06.163
[15]. Biris, A. R., Lazar, M. D., Pruneanu, S., Neamtu, C., Watanabe, F., Kannarpady, G. K., Dervishi, E., & Biris, A. S. (2013). Catalytic one-step synthesis of PT-decorated few-layer graphenes. RSC Advances, 3(48), 26391. https://doi.org/10.1039/c3ra44564j
[16]. Lazar, M. D., Biris, A. R., Borodi, G., Voica, C., Watanabe, F., Dervishi, E., & Biris, A. S. (2013). Magnesia supported au and ag catalysts for the preparation of few-layer graphene-metal nanocomposites: Relationship between catalyst structure and the properties of graphene composites. Journal of Materials Science, 48(21), 7409-7421. https://doi.org/10.1007/s10853-013-7556-6
[17]. Biris, A. R., Dervishi, E., Ardelean, S., Lazar, M. D., Watanabe, F., Biris, G. L., Misan, I., & Biris, A. S. (2013). Synthesis of AG-decorated, few-layer graphene structures over a novel AG/mgo catalytic system by radio-frequency chemical vapor deposition. Materials Chemistry and Physics, 138(2-3), 454-461. https://doi.org/10.1016/j.matchemphys.2012.11.030
[18]. Dervishi, E., Biris, A. R., Driver, J. A., Watanabe, F., Bourdo, S., & Biris, A. S. (2013). Low-temperature (150° C.) carbon nanotube growth on a catalytically active iron oxide-graphene nano-structural system. Journal of Catalysis, 299, 307-315. https://doi.org/10.1016/j.jcat.2012.12.013
[19]. Biris, A. R., Ardelean, S., Lazar, M. D., Dervishi, E., Watanabe, F., Ghosh, A., Biswas, A., & Biris, A. S. (2012). Synthesis of few-layer graphene over gold nanoclusters supported on mgo. Carbon, 50(6), 2252-2263. https://doi.org/10.1016/j.carbon.2012.01.043
[20]. Dervishi, E., Biris, A. R., Watanabe, F., Umwungeri, J. L., Mustafa, T., Driver, J. A., & Biris, A. S. (2011). Few-layer nano-graphene structures with large surface areas synthesized on a multifunctional fe:mo:mgo catalyst system. Journal of Materials Science, 47(4), 1910-1919. https://doi.org/10.1007/s10853-011-5980-z
[21]. Xu, Y., Dervishi, E., Biris, A. R., & Biris, A. S. (2011). Chirality-enriched semiconducting carbon nanotubes synthesized on high surface area mgo-supported catalyst. Materials Letters, 65(12), 1878-1881. https://doi.org/10.1016/j.matlet.2011.03.040
[22]. Biris, A. R., Dervishi, E., Simon, S., Lupu, D., Misan, I., Iancu, C., Valeria Clichici, S., Xu, Y., Watanabe, F., & Biris, A. S. (2010). Analytic studies of high quality singlewall carbon nanotubes synthesized on a novel Fe:mo:mgo catalyst. Physica E: Low-Dimensional Systems and Nanostructures, 43(1), 552-558. https://doi.org/10.1016/j.physe.2010.09.014
[23]. Wayland, H. A., Boury, S. N., Chhetri, B. P., Brandt, A., Proskurnin, M. A., Filichkina, V. A., Zharov, V. P., Biris, A. S., Ghosh, A. (2016). Advanced cellulosic materials for treatment and detection of industrial contaminants in wastewater. ChemistrySelect, 1(15), 4472-4488. https://doi.org/10.1002/slct.201600653
[24]. Jenkins, S. V., Nima, Z. A., Vang, K. B., Kannarpady, G., Nedosekin, D. A., Zharov, V. P., Griffin, R. J., Biris, A. S., Dings, R. P. (2017). Triple-negative breast cancer targeting and killing by EPCAM-directed, plasmonically active nanodrug systems. Npj Precision Oncology, 1(1). https://doi.org/10.1038/s41698-017-0030-1
[25]. Karmakar, A., Bratton, S. M., Dervishi, E., Ghosh, A., Mahmood, M., Xu, Y., Saeed, L. M., Mustafa, T., Casciano, D., Radominska-Pandya, A., & Biris, A. S. (2011). Ethylenediamine functionalized-single-walled nanotube (f-SWNT)-assisted in vitro delivery of the oncogene suppressor p53 gene to breast cancer MCF-7 cells. International journal of nanomedicine, 6, 1045-1055. https://doi.org/10.2147/IJN. S17684
[26]. Biris, A. R., Toloman, D., Popa, A., Lazar, M. D., Kannarpady, G. K., Saini, V., Watanabe, F., Chhetri, B. P., Ghosh, A., Biris, A. S. (2016). Synthesis of tunable core-shell nanostructures based on tio2-graphene architectures and their application in the photodegradation of Rhodamine Dyes. Physica E: Low-Dimensional Systems and Nanostructures, 81, 326-333. https://doi.org/10.1016/j.physe.2016.03.028
[27]. Parnell, C. M., Chhetri, B. P., Mitchell, T. B. et al. Simultaneous Electrochemical Deposition of Cobalt Complex and Poly(pyrrole) Thin Films for Supercapacitor Electrodes. Sci Rep 9, 5650 (2019). https://doi.org/10.1038/s41598-019-41969-6
[28]. Chhetri, B. P., Parnell, C. M., Wayland, H., RanguMagar, A. B., Kannarpady, G., Watanabe, F., Albkuri, Y. M., Biris, A. S., Ghosh, A. (2018). Chitosan-derived nio-mn 2 O 3/C nanocomposites as non-precious catalysts for enhanced oxygen reduction reaction. ChemistrySelect, 3(3), 922-932. https://doi.org/10.1002/slct.201702907
[29]. P Navarri, D Marchal, A Ginestet, Activated carbon fibre materials for VOC removal, Filtration & Separation, Volume 38, Issue 1, 2001, Pages 33-40, ISSN 0015-1882, https://doi.org/10.1016/S0015-1882(01)80150-6.
[30]. Karmakar, A., Bratton, S. M., Dervishi, E., Ghosh, A., Mahmood, M., Xu, Y., Saeed, L. M., Mustafa, T., Casciano, D., Radominska-Pandya, A., & Biris, A. S. (2011). Ethylenediamine functionalized-single-walled nanotube (f-SWNT)-assisted in vitro delivery of the oncogene suppressor p53 gene to breast cancer MCF-7 cells. International journal of nanomedicine, 6, 1045-1055. https://doi.org/10.2147/IJN. S17684.
[31]. Ghosh, A., Ramidi, P., Pulla, S., Sullivan, S. Z., Collom, S. L., Gartia, Y., Munshi, P., Biris, A. S., Noll, B. C., & Berry, B. C. (2010). Cycloaddition of CO2 to epoxides using a highly active CO(III) complex of tetraamidomacrocyclic ligand. Catalysis Letters, 137(1-2), 1-7. https://doi.org/10.1007/s10562-010-0325-0
[32]. Ramidi, P., Munshi, P., Gartia, Y., Pulla, S., Biris, A. S., Paul, A., & Ghosh, A. (2011). Synergistic effect of alkali halide and Lewis base on the catalytic synthesis of cyclic carbonate from CO2 and epoxide. Chemical Physics Letters, 512(4-6), 273-277. https://doi.org/10.1016/j.cplett.2011.07.035
[33]. https://www.assemblymag.com/articles/94275-sterilizing-medical-devices-requires-friendly-agents
[34]. https://oaktrust.library.tamu.edu/bitstream/handle/1969.1/187191/MUSCHALEK-THESIS-2017.pdf;jsessionid=297A672AAE175CCEF0120F90E9CC4C9F?sequence=1
[35]. Haim Zada, M., Kumar, A., Elmalak, O., Mechrez, G., & Domb, A. J. (2019). Effect of Ethylene Oxide and Gamma (γ-) Sterilization on the Properties of a PLCL Polymer Material in Balloon Implants. ACS omega, 4(25), 21319-21326. https://doi.org/10.1021/acsomega.9b02889.
[36]. Karmakar, A., Bratton, S. M., Dervishi, E., Ghosh, A., Mahmood, M., Xu, Y., Saeed, L. M., Mustafa, T., Casciano, D., Radominska-Pandya, A., & Biris, A. S. (2011). Ethylenediamine functionalized-single-walled nanotube (f-SWNT)-assisted in vitro delivery of the oncogene suppressor p53 gene to breast cancer MCF-7 cells. International journal of nanomedicine, 6, 1045-1055. https://doi.org/10.2147/IJN. S17684
[37]. Pruneanu, S., Biris, A. R., Pogacean, F., Lazar, D. M., Ardelean, S., Watanabe, F., Dervishi, E., & Biris, A. S. (2012). Novel multifunctional graphene sheets with encased au/ag nanoparticles for advanced electrochemical analysis of organic compounds. ChemPhysChem, 13(16), 3632-3639. https://doi.org/10.1002/cphc.201200424
[38]. Gunawan, G., Bourdo, S., Biris, A. S., & Viswanathan, T. (2013). Microwave-assisted synthesis of highly dispersed carbon supported nickel nanoparticles (NI/C) with Renewable Carbon Source. Synthesis and Reactivity in Inorganic, Metal-Organic, and Nano-Metal Chemistry, 43(5), 635-639. https://doi.org/10.1080/15533174.2012.749898
[39]. Nedosekin D A, Galanzha E I, Dervishi E, Biris A S, Zharov V P. Super-resolution nonlinear photothermal microscopy. Small. 2014 Jan. 15; 10(1):135-42. https://doi.org/10.1002/smll.201300024. Epub 2013 Jul. 17. PMID: 23864531.
[40]. Biris, A. R., Lazar, M. D., Pruneanu, S., Neamtu, C., Watanabe, F., Kannarpady, G. K., Dervishi, E., Biris, A. S. (2013). Catalytic one-step synthesis of PT-decorated few-layer graphenes. RSC Advances, 3(48), 26391. https://doi.org/10.1039/c3ra44564j.
[41]. RanguMagar, A. B., Chhetri, B. P., Parameswaran-Thankam, A., Watanabe, F., Sinha, A., Kim, J.-W., Saini, V., Biris, A. S., Ghosh, A. (2018). Nanocrystalline cellulose-derived doped carbonaceous material for rapid mineralization of nitrophenols under Visible light. ACS Omega, 3(7), 8111-8121. https://doi.org/10.1021/acsomega.8b01020
[42]. Wilson A T, Bruno P. The sterilization of bacteriological media and other fluids with ethylene oxide. J Exp Med. 1950 May 1; 91(5):449-58.
[43]. Collins J. L. “Epoxy compounds”. Encyclopedia of the ILO. Institute of Industrial Safety, Labour Protection and Social Partnership. Retrieved 25 Sep. 2009. “Vol. 60. Some Industrial Chemicals”. IARC Monographs on the Evaluation of Carcinogenic Risks to Humans. Lyon: International Agency for Research on Cancer. 1999. ISBN 978-92-832-1297-3. Archived from the original on 3 Mar. 2016. Retrieved 28 Jun. 2007.
[44]. Glaser Z R. Ethylene oxide: toxicology review and field study results of hospital use. J Environ Pathol Toxicol. 1979 May-June; 2(5):173-208.
[45]. Lynch H N, Kozal J S, Russell A J, Thompson W J, Divis H R, Freid R D, Calabrese E J, Mundt K A. Systematic review of the scientific evidence on ethylene oxide as a human carcinogen. Chem Biol Interact. 2022 Sep. 1; 364:110031.

What is claimed is:

1. An apparatus for converting a toxic gas to benign substances, comprising:
a housing characterized with multi-stages including a first stage, a second stage, a third stage and a fourth stage coupled to one another in sequence, wherein
the first stage comprises a catalytic system configured to convert the toxic gas into byproducts;
the second stage comprises a carbonaceous fibrous material adapted to capture the remaining toxic gas and the byproducts;
the third stage comprises at least one oxidizer to oxidize the remaining toxic gas to benign substances including CO₂ and water; and
the fourth stage comprises a scrubber configured to remove all of volatile organic compounds or water molecules generated as part of the first and third stages.

2. The apparatus of claim 1, wherein the catalytic system comprises metal catalysts, metal oxide catalysts, supported on metal oxides, zeolites, graphitic materials, Lewis acidic catalysts, bases such as amines, halides, acetates, oxides, nitrites, ammonium, and/or phosphonium.

3. The apparatus of claim 2, wherein the catalytic system comprises metallic or oxides of Co, Fe, Co/Fe, Ni, Co/Mo, Pt, W, supported over MgO, CaCO₃, CaO, graphitic nanostructures, SiO₂ with various compositions and ratios, wherein the ratios range from 0.001 to 99 wt. %.

4. The apparatus of claim 2, wherein the catalytic system is in the form of powders with an average size ranging from nanometers to centimeters, and/or beds of porosities with an average pore size ranging from nanometers to centimeters.

5. The apparatus of claim 4, wherein the catalytic system comprises an active porous catalytic bed formed of a metal oxide nanocluster supported on magnesia (MgO).

6. The apparatus of claim 5, wherein the metal oxide nanocluster comprises cobalt tetraoxide (Co₃O₄).

7. The apparatus of claim 6, wherein a ratio of Co₃O₄ to MgO is in a range from 0.01 wt % to 99.99 wt %.

8. The apparatus of claim 2, wherein the first stage further comprises carbon dioxide (CO₂) being introduced into the first stage at a temperature ranging from room temperature to over 100° C.

9. The apparatus of claim 1, wherein the carbonaceous fibrous material is decorated with amine functionalization configured to trap the remaining toxic gas and the derivatives through surface bonding.

10. The apparatus of claim 9, wherein the carbonaceous fibrous material is decorated with amine NH₂ functional chemical groups including primary and secondary amine groups pyridinic, imidazole, and/or with zinc oxide (ZnO) aluminum oxide (Al₂O₃) nanostructures, CaO, CaCO₃, MgO, Ti₂ and derivatives, SiO₂, or zeolites.

11. The apparatus of claim 10, wherein the carbonaceous fibrous material comprises carbon nanofibers having a surface area in a range of 1-200 m²/g, preferably in a range of 40-100 m²/g.

12. The apparatus of claim 11, wherein the second stage further comprises Lewis acidic catalysts including alumina or zinc salts, metal catalysts, metal oxide catalysts, supported on metal oxides, zeolites, graphitic materials, Lewis acids catalysts, bases such as amines, halides, acetates, oxides, nitrites, ammonium, and/or phosphonium.

13. The apparatus of claim 11, wherein the second stage further comprises polymeric, cellulose or other organic and inorganic porous systems that can be functionalized with chemical group functionalities, wherein the other organic and inorganic porous systems include chitosan, starch, Xanthan, alginate, polyvinyl alcohol, alumina, ZnO, SiO2, MgO, CaO, CaCo3, and/or zeolites.

14. The apparatus of claim 1, wherein the at least one oxidizer comprises a solid oxidizer including persulphate or perborate.

15. The apparatus of claim 14, wherein the third stage further comprises metal salts adapted to induce Fenton type chemistry along with the solid oxidizer.

16. The apparatus of claim 15, wherein the metal salts includes cobalt oxide or iron oxide or iron sulfate particles

17. The apparatus of claim 15, wherein the at least one oxidizer and the metal salts are made into a porous particulate bed supported over an alumina oxide structure.

18. The apparatus of claim 1, wherein the scrubber comprises high surface area activated charcoal or graphitic structures, carbon nanostructures of various shapes and sizes (graphene, fibers, nanotubes, plates), porous structures decorated with such materials, graphite, cellulose, Chitosan, starch, Xanthan, alginate, polyvinyl alcohol, and/or polyurethanes.

19. The apparatus of claim 18, wherein the fourth stage further comprises adsorbing media that capture the remaining toxic gas and the derivatives and can be functionalized with various functional chemical groups.

20. The apparatus of claim 19, wherein the adsorbing media comprise zeolites, graphitic materials, polymeric structures, metal/metal oxides, cellulose, Lewis acidic catalysts, organic or inorganic porous systems, or a combination of them.

21. The apparatus of claim 1, wherein the toxic gas comprises ethylene oxide, propylene oxide, ozone, nitric oxides, NOx, volatile organic carbons, CO, or sox.

22. A method for converting a toxic gas into benign substances, comprising:
catalytically converting the toxic gas into byproducts in a first stage;
capturing the remaining toxic gas and the byproducts in a second stage;
oxidizing the remaining toxic gas to benign substances including CO₂ and water in a third stage; and
removing, from a fourth stage, all of volatile organic compounds or water molecules generated as part of the first and third stages.

23. The method of claim 22, wherein the first stage comprises a catalytic system comprising metal catalysts, metal oxide catalysts, supported on metal oxides, zeolites, graphitic materials, Lewis acidic catalysts, bases such as amines, halides, acetates, oxides, nitrites, ammonium, and/or phosphonium.

24. The method of claim 23, wherein the catalytic system comprises metallic or oxides of Co, Fe, Co/Fe, Ni, Co/Mo, Pt, W, supported over MgO, CaCO₃, CaO, graphitic nanostructures, SiO₂ with various compositions and ratios, wherein the ratios range from 0.001 to 99 wt. %.

25. The method of claim 24, wherein the catalytic system is in the form of powders with an average size ranging from nanometers to centimeters, and/or beds of porosities with an average pore size ranging from nanometers to centimeters.

26. The method of claim 25, wherein the catalytic system comprises an active porous catalytic bed formed of a metal oxide nanocluster supported on magnesia (MgO).

27. The method of claim 26, wherein the metal oxide nanocluster comprises cobalt tetraoxide (Co₃O₄).

28. The method of claim 27, wherein a ratio of Co₃O₄ to MgO is in a range from 0.01 wt % to 99.99 wt %.

29. The method of claim 22, further comprising intruding carbon dioxide (CO₂) into the first stage at a temperature ranging from room temperature to over 100° C.

30. The method of claim 22, wherein the second stage comprises a carbonaceous fibrous material decorated with amine functionalization configured to trap the remaining toxic gas and the derivatives through surface bonding.

31. The method of claim 30, wherein the carbonaceous fibrous material is decorated with amine NH₂ functional chemical groups including primary and secondary amine groups pyridinic, imidazole, and/or with zinc oxide (ZnO) or aluminum oxide (Al₂O₃) nanostructures, CaO, CaCO₃, MgO, Ti₂ and derivatives, SiO₂, or zeolites.

32. The method of claim 31, wherein the carbonaceous fibrous material comprises carbon nanofibers having a surface area in a range of 1-200 m²/g, preferably in a range of 40-100 m²/g.

33. The method of claim 32, wherein the second stage further comprises Lewis acidic catalysts including alumina or zinc salts, metal catalysts, metal oxide catalysts, supported on metal oxides, zeolites, graphitic materials, Lewis acids catalysts, bases such as amines, halides, acetates, oxides, nitrites, ammonium, and/or phosphonium.

34. The method of claim 32, wherein the second stage further comprises polymeric, cellulose or other organic and inorganic porous systems that can be functionalized with chemical group functionalities, wherein the other organic and inorganic porous systems include chitosan, starch, Xanthan, alginate, polyvinyl alcohol, alumina, ZnO, SiO₂, MgO, CaO, CaCO₃, and/or zeolites.

35. The method of claim 22, wherein the third stage comprises at least one oxidizer comprising a solid oxidizer including persulphate or perborate.

36. The method of claim 35, wherein the third stage further comprises metal salts adapted to induce Fenton type chemistry along with the solid oxidizer.

37. The method of claim 36, wherein the metal salts includes cobalt oxide or iron oxide or iron sulfate particles

38. The method of claim 36, wherein the at least one oxidizer and the metal salts are made into a porous particulate bed supported over an alumina oxide structure.

39. The method of claim 22, wherein the fourth stage comprises a scrubber comprising high surface area activated charcoal or graphitic structures, carbon nanostructures of various shapes and sizes (graphene, fibers, nanotubes, plates), porous structures decorated with such materials, graphite, cellulose, chitosan, starch, Xanthan, alginate, polyvinyl alcohol, and/or polyurethanes.

40. The method of claim 39, wherein the fourth stage further comprises adsorbing media that capture the remaining toxic gas and the derivatives and can be functionalized with various functional chemical groups.

41. The method of claim 40, wherein the adsorbing media comprise zeolites, graphitic materials, polymeric structures, metal/metal oxides, cellulose, Lewis acidic catalysts, organic or inorganic porous systems, or a combination of them.

42. The method of claim 22, wherein the toxic gas comprises ethylene oxide, propylene oxide, ozone, nitric oxides, NOx, volatile organic carbons, CO, or sox.
